(19) United States
(12) Patent Application Publication
(10) Pub. No.: US 2008/0227710 A1
(43) Pub. Date: Sep. 18, 2008

(54) Use of Conjugates of Doxorubicin with Lactosaminated Albumin

(76) Inventors: Luigi Fiume, Bologna (IT); Giuseppina Di Stefano, San Lazzaro Di Savena (BO) (IT)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 12/067,211

(22) PCT Filed: Sep. 19, 2006
(86) PCT No.: PCT/EP06/66489

§ 371 Date: Mar. 18, 2008

(30) Foreign Application Priority Data
Sep. 20, 2005 (IT) MI2005A001743

(51) Int. Cl. A61K 38/38; A61P 35/00

(52) U.S. Cl. 514/12

(57) ABSTRACT

The present invention refers to the use of a conjugate of doxorubicin with lactosaminated human albumin for the preparation of a pharmaceutical composition useful in the treatment of hepatocellular carcinomas (HCCs) which do not express the asialoglycoprotein receptor (ASGP-R). The conjugate which was previously prepared and studied only for the treatment of HCCs expressing the ASGP-R, has now been shown to possess the potentiality of a beneficial use also in the treatment of the HCCs which do not have the receptor. Therefore, compositions containing the conjugate could be administered for treatment of all HCCs, without the need of a preliminary tumor biopsy to demonstrate the presence or the absence of the receptor.

The object of the present invention is the use of a conjugate of doxorubicin with lactosaminated human albumin for the preparation of a pharmaceutical composition useful in the treatment of hepatocellular carcinomas (HCCs) which do not express the asialoglycoprotein receptor (ASGP-R). The conjugate which was previously prepared and studied only for the treatment of HCCs expressing the ASGP-R, has now been unexpectedly shown to possess the potentiality for a beneficial use also in the treatment of the HCCs which do not have the receptor. Therefore, compositions containing the conjugate could be administered for treatment of all HCCs, without the need of a preliminary tumor biopsy to demonstrate the presence or the absence of the receptor. In previous experiments we coupled doxorubicin (DOXO) to lactosaminated human albumin (L-HSA) in order to increase the anticancer activity of the drug and reduce its toxic side effects in the treatment of those hepatocellular carcinomas whose cells express the receptor for asialoglycoproteins (ASGP-R) (Di Stefano G, et al. Doxorubicin coupled to lactosaminated human albumin . . . Dig Liver Dis 2003; 35: 428-433; Fiume L, et al. Doxorubicin coupled to lactosaminated albumin . . . J Hepatol 2005; 43: 645-652). HCCs are tumors resistant to chemotherapeutic agents; DOXO is active on HCCs, but displays severe adverse reactions at the effective doses (Llovet J M. Updated treatment . . . J Gastroenterol 2005; 40:225-235). Toxic effects of DOXO are mainly produced on heart, bone marrow and intestine (Mazue G, et al. Anthracyclines: a review . . . Int J Oncol 1995; 7: 713-26). The ASGP-R is present only on the surface of hepatocytes. It mediates uptake and lysosomal degradation of galactosyl terminating peptides (Ashwell G, et al. Carbohydrate-specific . . . Annu Rev Biochem 1982; 51: 531-534), which can be used as vectors for selectively delivering the drug to parenchymal liver cells (Fiume L, et al. Liver targeting of antiviral nucleoside analogs through the asialoglycoprotein receptor. J Viral Hep 1997; 4: 363-370). L-HSA is a galactosyl terminating neoglycoprotein that has been successfully used as hepatotropic drug carriers in humans (Torrani Cerenzia M R, et al. Adenine arabinoside monophosphate . . . Hepatology 1996; 23: 657-661; Zarski J P, et al. Efficacy and safety of L-HSA-ara-AMP . . . J Hepatol 2001; 34: 487-488). DOXO was coupled to L-HSA by an acid sensitive hydrazone bond that allows DOXO to be intracellularly released from the carrier in the endosomal and lysosomal compartments (Greenfield R S, et al. Evaluation in vitro of adriamycin . . . Cancer Res 1990; 50: 6600-6607). (The conjugation procedure is covered by a patent filed by the University of Bologna (PCT/IT2005/000257)). ASGP-R is maintained on the neoplastic cells of the majority of well differentiated (WD) human HCCs, whereas it is not expressed on the cells of the majority of the poorly differentiated (PD) HCCs (Hyodo I, et al. Distribution of asialoglycoprotein receptor . . . Liver 1993; 13: 80-85; Trerè D, et al. The asialoglycoprotein receptor . . . Br J Cancer 1999; 81: 404-408; Sawamura T, et al. “Hyperasialoglycoproteinemia in patients . . . ” Gastroenterology 1984; 87: 1217-1221). Presence or absence of the ASGP-R in human HCCs can be determined immunohistochemically using needle biopsies or fragments from surgically removed tumors. In rats with HCCs induced by diethylnitrosamine (DENA) the ability of the tumors to internalize L-HSA through the ASGP-R was found in all the 7 studied WD HCCs, in 4 out of 5 moderately differentiated (MD) tumors, whereas a low capacity to take up L-HSA was observed only in 2 out of 5 PD HCCs (Di Stefano G, et al. Enhanced uptake of lactosaminated . . . Liver Int 2005; 25: 854-860).

In experiments on the distribution of a radioactive L-HSA-DOXO in HCCs and organs of rats we made two unexpected and related observations:

1—PD HCCs internalize amounts of conjugate three times higher than those of L-HSA

2—In all the forms of HCCs, independently of their differentiation grade, the conjugate produced high concentrations of DOXO, 5-8 fold higher than those measured in intestine and heart.

### MATERIALS AND METHODS

Synthesis and Characterization of L[¹⁴C]HSA-DOXO

Human albumin (HSA) was obtained by Kedrion (Lucca, Italy). It was gel-filtered on Sephacryl S-200 HR (Sigma, St Louis, Mo., USA) and the monomer (>90% of the starting material) was collected and used. HSA was labeled with [¹⁴C]sucrose. [¹⁴C]-sucrose is a radioactive tracer which remains within the entered cells, allowing an exact determination of protein uptake in tissues (Pittman R C, et al. “Radiolabeled sucrose . . . ” J Biol Chem. 1979; 254: 6876-6879). [¹⁴C]-sucrose (500 mCi/mmol, Amersham, Buckinghamshire, UK) was diluted to a specific activity of 3.2×10⁵ dpm/μg. Coupling of [¹⁴C]-sucrose to HSA was performed according to Pittman R C et al. (“Radiolabeled sucrose . . . ” J Biol Chem. 1979; 254: 6876-6879). Since this procedure causes a protein oligomerization, labeled HSA was gel-chromatographed on a Sephacryl S200 HR column, and the monomer (70% of preparation) was collected. The molar ratio [¹⁴C]-sucrose/HSA was determined by counting the radioactivity and measuring the protein according to Lowry O H et al. (“Protein measurement . . . ” J Biol Chem 1951; 193: 265-275)—and it was 0.2. Alpha-lactose (Sigma) was coupled to [¹⁴C]HSA by reductive amination (Wilson G. “Effect of reductive lactosamination . . . ” J Biol Chem 1978; 253: 2070-2072). The molar ratio lactose/[¹⁴C]HSA was determined by measuring the sugar according to Dubois M et al. (“Colorimetric method for determination . . . ” Anal Chem 1956; 28: 350-356) and it was 26. Coupling of DOXO to L-[¹⁴C]HSA was performed using the (6-maleimidocaproyl)hydrazone derivative of the drug (DOXO-EMCH), synthesized according to Willner D, et al. (“(6-maleimidocaproyl)hydrazone derivative . . . ” Bioconj Chem 1993; 4: 521-527). Coupling of DOXO-EMCH with L-[¹⁴C]HSA was carried out according to Di Stefano G, et al. (“A novel method for coupling . . . ” Eur J Pharm Sci 2004; 23: 393-397), here attached for reference, but allowing the compounds to react at 200. The molar ratio DOXO/L-[¹⁴C]HSA were calculated by measuring the protein concentration according to Lowry O H, et al. (Protein measurement . . . J Biol Chem 1951; 193: 265-275) and DOXO by absorbance at λ₄₉₅ [ε₄₉₅(pH 7.4) of DOXO-EMCH=9250 M⁻¹ cm⁻¹]. It was 5.8 (1 mg DOXO was contained in 24 mg conjugate; 24 mg conjugate contained 22.5 mg L-HSA).

Induction of HCCs in Rats; Distribution of the Conjugate and DOXO Concentrations in Tumors and Organ.

Male Wistar rats were used. They were obtained from Harlan Italy (Udine, Italy) and were maintained in an animal facility at the Department of Experimental Pathology, Bologna. The protocols of the experiments were approved by the Ethical Committee of the University of Bologna.

HCCs were induced by diethyInitrosamine (DENA) given in the drinking water (100 mg/l) for 8 weeks. Six to eight weeks after the last day of DENA administration, animals were i.v. injected with the following compounds: L-[¹⁴C]HSA (22.5 μg/g), L-[¹⁴C]HSA-DOXO (24 μg/g, corresponding to 22.5 μg/g of L-[¹⁴C]HSA and to 1 μg/g of DOXO) and free DOXO (1 μg/g). Compounds were injected in the dorsal vein of penis, in a volume of 10 μl/10 g body weight, under isoflurane anaesthesia. For each compound four rats were used. Four hours after the injection the animals were killed under isoflurane anaesthesia. Organs were rapidly removed and frozen and neoplastic nodules were accurately dissected from the surrounding liver. A part of each tumor nodule was frozen; the other part was fixed in 10% formalin and processed for histology. Samples of frozen organs and nodules were used for radioactivity count and determination of DOXO levels. DOXO was measured according to Bots A M, et al. (Analysis of adriamycin . . . J Chromatogr 1983; 272: 421-427), with modifications (Di Stefano G, et al. Doxorubicin coupled to lactosaminated . . . Dig Liver Dis 2003; 35: 428-433).

Results

In rats, DENA induced well, moderately and poorly differentiated forms of HCCs (WD, MD, and PD HCCs, respectively). They showed histological features super-imposable to those of human HCCs. As described by Di Stefano G et al. (“Enhanced uptake of lactosaminated . . . ” Liver Int 2005; 25: 854-860) in WD HCCs neoplastic hepatocytes were isomorphic with eosinophilic cytoplasm; they were similar to their non-neoplastic counterpart with a trabecular pattern and intervening vascular spaces. In PD HCCs the tumor tissue showed a solid appearance; neoplastic cells were polymorphic with a basophilic cytoplasm. The MD HCCs showed a histological appearance intermediate between that of WD and that of PD HCCs. Distribution of L-[¹⁴C]HSA and of L-[¹⁴C]HSA-DOXO in HCCs, heart and intestine is reported in Table 1. In agreement with Di Stefano G et al. (“Enhanced uptake of lactosaminated . . . ” Liver Int 2005; 25: 854-860), in four out of six examined PD HCCs, the L-[¹⁴C]HSA reached concentrations not higher than those measured in heart and intestine, organs which do not express the ASGP-R (dpm/g/SA=9.1±0.1). The conjugate L-[¹⁴C]HSA-DOXO was internalized by PD HCCs in amounts four times higher than those of L-[¹⁴C]HSA (p=0.004, according to the Student's t-test) and entered in all the seven examined PD HCCs in quantities at least seven times higher than those taken up by heart and intestine.

An important finding was that in animals injected with the conjugate DOXO concentrations were more than eight and five times higher than those measured in heart and intestine, respectively (p=0.006). On the contrary, in animals injected with the free drug the DOXO concentrations measured in heart and intestine were higher than those determined in HCCs (Table 2).

In conclusion, contrary to unconjugated DOXO, L-HSA-DOXO produces in all HCCs, drug concentrations higher than those in heart and intestine, target organs of toxic action of DOXO, independently of the differentiation grade of the tumors and their capacity of internalizing L-HSA. As a consequence, the conjugate, formerly prepared to increase the antineoplastic efficacy and to reduce the toxicity of DOXO in the treatment of HCCs that maintain the ability of internalizing proteins exposing galactosyl residues, on the basis of the present observations, shows the potentiality for improving the chemotherapeutic index of DOXO in the treatment of all HCC forms, including the poorly differentiated ones, which display no or only poor capacity to accumulate L-HSA with respect to extra-hepatic tissues.

Therefore, the object of the present invention is the use of L-SA-DOXO and particularly of L-HSA-DOXO in the treatment of all HCCs, independently of ASGP-R expression on their cells. Therefore, the grant of a patent is required for an use of the L-SA-DOXO conjugate in a chemotherapy of HCCs that does not require the preliminary search for the presence or the absence of ASGP-R and consequently avoids the need of a tumor biopsy with the related risks. Moreover, the patent claims the use of L-SA-DOXO conjugate in the chemotherapy of HCCs not expressing the ASGP-R.

TABLE 1
Distribution of L-[¹⁴C]HSA and L-[¹⁴C]HSA-DOXO in HCCs with
different differentiation grade, in heart and intestine

| | dpm/g/SA^{a)} | | | | |
|---|---|---|---|---|---|
| Compound | WD HCCs | MD HCCs | PD HCCs | Heart | Intestine |
| L-[¹⁴C]HSA^{b)} | 178.3 ± 12.4 | 85.6 ± 4.4 | 32.6 ± 14.9 | 8.7 ± 0.9 | 8.9 ± 1.5 |
| | (6)^{d)} | (5) | (6) | | |
| L-[¹⁴C]HSA-DOXO^{c)} | 180.9 ± 27.8 | 135.5 ± 19.3 | 124.8 ± 20.2 | 9.0 ± 0.8 | 9.8 ± 0.8 |
| | (7) | (10) | (7) | | |

Experimental details are described in Materials and Methods. Rats were killed 4 h after i.v. injection of compounds. For each compound, 4 animals were used. Data are mean values ± standard error.
^{a)}SA = Specific Activity
^{b)}L-[¹⁴C]HSA was injected at the dose of 22.5 μg/g.
^{c)}L-[¹⁴C]HSA-DOXO was injected at the dose of 24 μg/g (24 μg of conjugate contain 22.5 μg of L-[¹⁴C]HSA and 1 μg of DOXO).
^{d)}Number of examined HCCs

TABLE 2
DOXO concentrations in HCCs with different differentiation grade, in
heart and intestine measured after administration of
L-[¹⁴C]HSA-DOXO and of free DOXO

| | nmoles DOXO/g^{a)} | | | | |
|---|---|---|---|---|---|
| Compound | WD HCCs | MD HCCs | PD HCCs | Heart | Intestine |
| L-[¹⁴C]HSA- | 10.2 ± 2.0 | 7.6 ± 0.9 | 6.8 ± 1.1 | 0.8 ± 0.1 | 1.2 ± 0.2 |
| DOXO^{b)} | (9)^{d)} | (14) | (8) | | |
| DOXO^{c)} | 3.4 ± 0.6 | 3.1 ± 0.4 | 3.6 ± 0.4 | 4.7 ± 0.3 | 4.2 ± 0.6 |
| | (5) | (4) | (8) | | |

Experimental details are described in Materials and Methods. Data are mean values ± standard error.
^{a)}In animals injected with the conjugate DOXO was measured as free drug. i.e. liberated from the carrier L-HSA inside the cells.
^{b)}L-[¹⁴C]HSA-DOXO was injected at the dose of 24 μg/g (24 μg of conjugate contain 1 μg of DOXO).
^{c)}DOXO was injected at the dose of 1 μg/g.
^{d)}Number of examined HCCs.

1-8. (canceled)

9. A method for treating hepatocellular carcinomas which comprises administering to a patient a conjugate of doxorubicin with lactosaminated albumin, which method does not comprise the preliminary demonstration of the presence or of the absence of the receptor for the asialoglycoproteins in the tumoral cells.

10. A method according to claim 9, wherein said lactosaminated albumin is lactosaminated human albumin.

11. A method according to claim 9, wherein said pharmaceutical composition is administrable by parenteral route.

12. A method according to claim 11, wherein said pharmaceutical composition is administrable by intravenous route, by bolus or by infusion.

13. A method according to claim 9, wherein said pharmaceutical composition is an aqueous solution.

14. A method according to claim 13, wherein said aqueous solution contains excipients and/or pharmaceutically acceptable coadjuvants.

15. A method according to claim 9, wherein said patient is a human.

16. A method for treating hepatocellular carcinomas which do not express the receptor for the asialoglycoproteins, which comprises administering to a patient a conjugate of doxorubicin with lactosaminated albumin.

17. A method according to claim 16, wherein the administration of the conjugate does not comprise the preliminary demonstration of the presence or of the absence of the receptor for the asialoglycoproteins in the tumour cells.

18. A method according to claim 16, wherein said lactosaminated albumin is lactosaminated human albumin.

19. A method according to claim 16, wherein said pharmaceutical composition is administrable by parenteral route.

20. A method according to claim 19, wherein said pharmaceutical composition is administrable by intravenous route, by bolus or by infusion.

21. A method according to claim 16, wherein said pharmaceutical composition is an aqueous solution.

22. A method according to claim 21, wherein said aqueous solution contains excipients and/or pharmaceutically acceptable coadjuvants.

23. A method according to claim 16, wherein said patient is a human.
